(19) United States
(12) Patent Application Publication
(10) Pub. No.: US 2013/0303490 A1
(43) Pub. Date: Nov. 14, 2013

(54) Inhibitors of HIV Infections and uses thereof

(71) Applicants: Vincent Francois, Montpellier (FR); Pierre Corbeau, Montpellier (FR); Charline Duquenne, Montpellier (FR)

(72) Inventors: Vincent Francois, Montpellier (FR); Pierre Corbeau, Montpellier (FR); Charline Duquenne, Montpellier (FR)

(73) Assignee: Centre National De La Recherche Scientifique-CNRS, Paris (FR)

(21) Appl. No.: 13/916,764

(22) PCT Filed: Dec. 12, 2011
(86) PCT No.: PCT/FR2011/052948

§ 371 Date: Jun. 13, 2013

(30) Foreign Application Priority Data
Dec. 13, 2010 (FR) 10 60432

(51) Int. Cl. A61K 31/661; A61K 31/137; A61K 31/4245

(52) U.S. Cl. CPC A61K 31/661; A61K 31/4245; A61K 31/137
USPC 514/114; 514/364; 514/653; 558/169; 548/131; 564/360

(57) ABSTRACT

The invention relates to compounds which are agonists of at least one receptor selected from S1PR1, S1PR2, S1PR3, S1PR4 and S1PR5 receptors, for use in methods for treating HIV infections in humans or animals.

### FIELD OF THE INVENTION

The invention relates to compounds which are agonists of a receptor selected from S1PR1, S1PR2, S1PR3, S1PR4 and S1PR5 receptors, for use in the treatment of HIV infections.

### INTRODUCTION

The human immunodeficiency virus (HIV) is a retrovirus which infects human beings and is responsible for acquired immunodeficiency syndrome (AIDS), which is a weakened state of the immune system, making it vulnerable to multiple opportunistic infections.

AIDS, which is transmitted by several body fluids: blood, vaginal secretions, sperm or maternal milk, is today considered to be a pandemic having caused the death of approximately 25 million people between 1981 (date of the first identification of a case of AIDS) and January 2006. It is estimated that approximately 1% of people between the ages of 15 and 49 live with HIV, mainly in sub-Saharan Africa. Although there are antiretroviral treatments which combat HIV and consequently delay the appearance of AIDS, thus reducing mortality and morbidity, there is at the present time no vaccine or definitive treatment.

There is therefore a need for new molecules for effectively treating HIV infections.

### SUMMARY OF THE INVENTION

HIV penetrates into a target cell via binding of the gp120 protein of the viral envelope with the CD4 receptor, this binding then enables the formation of a ternary complex with the CCR5 coreceptor (R5-tropic virus or HIV-R5). This results in fusion of the viral envelope via the gp41 protein, with the cell membrane, which allows entry of the virus. CCR5 is a chemokine receptor present on various types of lymphocytes, and belongs to the G protein-coupled receptor (GPCR) family. Since CCR5 is the main coreceptor of HIV, it represents a particular physiopathological interest and a therapeutic target of choice.

The inventors have been able to show that the S1PR1 receptor heterodimerizes with the CCR5 coreceptor and that it reduces HIV infectivity. The inventors have also shown that S1PR1 receptor agonists can reduce the infection of cells by HIV. Following lengthy and thorough studies, the inventors have also shown that S1PR1, S1PR2, S1PR3, S1PR4 and S1PR5 receptor agonists are capable of reducing HIV infection.

Without wishing to be bound by any theory, the inventors envision several hypotheses for explaining the functioning of the agonists according to the invention.

A first hypothesis is that the agonist, by binding to an S1PR receptor, would promote the internalization of the S1PR1-CCR5 heterodimer, as has been proposed for the S1PR1 receptor alone, by Markus H. Gräler in Cell Physiol. Biochem 2010; 26: 79-86, in order to explain the immunomodulation induced by the FTY720-P agonist. This internalization of the S1PR1-CCR5 heterodimer would consequently reduce the amount of CCR5 coreceptor at the surface of the cell, thus explaining the decrease in HIV infection.

A second hypothesis is that the stimulation of an S1PR receptor induced by the binding of the agonist would reduce the signaling of the CCR5 receptor. This phenomenon, known as heterodesensitization, is well known. Thus, the activation of an S1PR receptor could, through its “inhibitory” effect, on CCR5, influence HIV infection and propagation. Such an anti-HIV effect has in particular been documented in the cases of CXCR1 stimulation by interleukin 8 (Richardson et al., 2003. J Biol Chem 278:15867-15873), of FPR stimulation by the bacterial peptide fMLF (Shen, W. et al., 2000. Blood 96:2887-2894), and of A2A stimulation by adenosine (Zhang et al., 2006. Blood 108:38-44).

A third hypothesis is that the stimulation of an S1PR receptor induced by the binding of the agonist would modify the interaction of CCR5 with CD4 or gp120, which would lead to a decrease in the infection of the cells by the virus.

Finally, in the light of results obtained using HIV-1 virus in which the envelope is replaced with that of the vesicular stomatitis virus (VSVG) and which therefore penetrates into the cell by endocytosis and independently of CCR5, the inventors have put forward a fourth hypothesis. The latter is based on the possibility that the signaling induced by the binding of an agonist to its S1PR receptor would stimulate the transduction pathway of this receptor in a way which interferes with the replicative cycle of HIV via a CCR5 independent mechanism.

The invention therefore relates to compounds which are S1PR1 receptor agonists, for use in methods for treating HIV infections in humans or animals.

### DETAILED DESCRIPTION OF THE INVENTION

According to the invention, the expression “sphingosine-1-phosphate receptor agonist” or “agonist of an S1PR receptor”, is intended to mean a compound which binds to at least one sphingosine-1-phosphate receptor selected from S1PR1, S1PR2, S1PR3, S1PR4 and S1PR5.

More specifically, the expression “S1PR1 receptor agonist” is intended to mean a compound which binds to the S1PR1 receptor and which, for example, leads to the internalization of the receptor, and/or the dissociation of the intracellular G protein heterotrimer into Gα-GTP and Gβγ, and/or an increase in the phosphorylation of the receptor, and/or the activation of the signaling pathway of the receptor. This term encompasses “complete” agonists and also “partial” agonists of the sphingosine-1-phosphate receptor.

In the subsequent text, the expressions “S1PR1 receptor” and “S1P1 receptor” are used without distinction.

According to the invention, the term “treatment” or “treating” is intended to mean the action of suppressing, reducing, or inhibiting the progression, or preventing the appearance, of the condition or of the disease to which this term applies; or the action of suppressing, reducing, or inhibiting the progression, or preventing the appearance, of one or more symptoms of the condition or of the disease to which this term applies.

According to the invention, the expression “HIV infection” is intended to mean an infection by any type, group or clade of human immunodeficiency virus (HIV). Indeed, HIV is a virus which has a very high genetic variability and thus exhibits a very great diversity. HIV may be of two types: HIV-1, the most widely present throughout the world, or HIV-2, which is less pathogenic than HIV-1. It comprises HIV-2A and HIV-2B. Within each type there are several groups which, in turn, comprise clades. HIV-1 is classified into four groups: group M (for major group), group O (for outlier group), group N (for non-M, non-O group) and group P. In 2005, group M widely predominated with more than 40 million contaminated individuals, compared with a little more than 500 for group O and only 7 for group N. Group M comprises nine subtypes or clades (from A to D, from F to H, J and finally, K). Added to this are several recombinant forms (“circulating recombinant form” or CRF), the origin of which is the multiple infection of a cell by different subtypes, thereby leading to mixtures in the viral genome.

Sphingosine-1-phosphate Receptor (S1PR) Agonists

The invention relates to a compound which is an agonist of at least one sphingosine-1-phosphate receptor, for use in methods for treating HIV infections in humans or animals.

Thus, the invention relates to a compound which is an agonist of at least one receptor selected from S1PR1, S1PR2, S1PR3, S1PR4 and S1PR5 receptors, for use in a method for treating an HIV infection in humans or animals.

S1PR1 (or S1P1) has a sequence of 382 amino acids, which is accessible under accession number NP_{—}001391.2 on the NCBI database. This sequence is represented by SEQ ID N° 1.

S1PR2 (or S1P2) has a sequence of 353 amino acids, which is accessible under accession number NP_{—}001391 on the NCBI database. This sequence is represented by SEQ ID N° 2.

S1PR3 (or S1P3) has a sequence of 378 amino acids, which is accessible under accession number NP_{—}005217 on the NCBI database. This sequence is represented by SEQ ID N° 3.

S1PR4 (or S1P4) has a sequence of 384 amino acids, which is accessible under accession number NP_{—}003766 on the NCBI database. This sequence is represented by SEQ ID N° 4.

S1PR5 (or S1P5) is present in the form of two isoforms.

The first isoform has a sequence of 310 amino acids, which is accessible under accession number AAH67781 on the NCBI database. This sequence is represented by SEQ ID N° 5.

The second isoform has a sequence of 398 amino acids, which is accessible under accession number NP_{—}001159687 on the NCBI database. This sequence is represented by SEQ ID N° 6.

The sphingosine-1-phosphate receptor (S1PR) agonists are known in the prior art and those skilled in the art have sufficient general knowledge to identify compounds which are agonists of an S1PR receptor. To do this, they can carry out an in vitro functional ³⁵S-GTPγS binding test. They may, for example, refer to the publication D S. Im et al., Mol. Pharmacol. 2000; 57-753. In this functional test, the binding between the GTPγS and the G proteins, mediated by the ligand, is measured in a GTP binding buffer (in mM: 50 HEPES; 100 NaCl, 10 MgCl₂, pH 7.5) using 25 μg of a membrane prepared from transiently transfected HEK293 cells. The ligand is added to the membranes in the presence of 10 μM of GDP and of 0.1 nM of ³⁵S-GTPγS (1200 Ci/mmol) and incubated at 30° C. for 30 minutes. The bound GTPγS is separated from the unbound GTPγS using a Brandel collector (Gaithersburg, Md.), and counted with a liquid scintillant counter. Typically, the compound according to the invention is selected from the group comprising sphingosine-1-phosphate, FTY720, FTY720-P, AUY954, CYM-5442, CYM-5181, SEW2871, VPC01091, DS-SG-44, KRP-203-P, DihydroS1P, Compound 26, Compound 12, sphingosylphosphorylcholine and AFD-R.

In another embodiment, the invention relates to a compound which is an agonist of at least two receptors selected from S1PR1, S1PR2, S1PR3, S1PR4 and S1PR5 receptors, for use in a method for treating an HIV infection in humans or animals.

Typically, said compound is an agonist of at least:
S1PR1 and S1PR2 receptors; or
S1PR1 and S1PR3 receptors; or
S1PR1 and S1PR4 receptors; or
S1PR1 and S1PR5 receptors; or
S1PR2 and S1PR3 receptors; or
S1PR2 and S1PR4 receptors; or
S1PR2 and S1PR5 receptors; or
S1PR3 and S1PR4 receptors; or
S1PR3 and S1PR5 receptors; or
S1PR4 and S1PR5 receptors.

In this embodiment, the compound according to the invention is selected from the compounds mentioned in the table below:

| | S1PR1 | | S1PR3 | | S1PR5 |
|---|---|---|---|---|---|
| | AGO- | S1PR2 | AGO- | S1PR4 | AGO- |
| COMPOUNDS | NIST | AGONIST | NIST | AGONIST | NIST |
| Sphingosine-1- | X | X | X | X | X |
| phosphate | | | | | |
| FTY720-P | X | | X | X | X |
| AUY954 | X | | X | | X |
| VPC01091 | X | | | X | X |
| DS-SG-44 | X | X | X | | |
| KRP-203-P | X | | | X | |
| DihydroS1P | X | X | X | X | X |
| Compound 26 | X | | X | X | X |
| Compound 12 | | | | X | X |
| Sphingosyl- | X | X | X | X | X |
| phosphorylcholine | | | | | |
| AFD-R | X | | X | X | X |

### S1PR1 Agonists

In one preferred embodiment, said agonist is an S1PR1 receptor agonist. Typically, it is a low-molecular weight agonist, for example, a small (possibly natural) organic molecule. The term “small organic molecule” refers to a molecule, which is possibly natural, having a size comparable to that of the organic molecules generally used as medicaments. This term excludes macromolecules (for example proteins, nucleic acid molecules, etc.). Preferred small organic molecules have a size of at most 10 000 Da, preferably of at most 5000 Da, more preferentially of at most 2000 Da, and even more preferentially of at most 1000 Da.

The sphingosine-1-phosphate receptors are known. Among these receptors, S1PR1 or “Sphingosine-1-phosphate receptor” is widely described in the literature. It is a receptor which belongs to the G protein-coupled receptor family, the natural ligand of which is sphingosine-1-phosphate (S1P). S1PR1 is also known as the following: EDG1, S1P1, ECGF1, EDG-1, CHEDG1, D1S3362, and FLJ58121. It is expressed ubiquitously and the genetic deletion thereof in mice has demonstrated its key role in angiogenesis and in vascular maturation, and also in the regulation of immune cell recruitment (Takabe et al., Pharmacol Rev 2008 June; 60(2):181-195). The involvement of sphingosine-1-phosphate in cancer has also been described by Weng In Leong et al., in Biochimie 92 (2010) 716-723.

The S1PR1 receptor agonists are also widely described in many scientific reviews. Mention may in particular be made of the review by Dong-Soon I M published in Acta Pharmacologica Sinica (2010) 31:1213-1222. Mention may also be made of patent applications EP1905434A1 or WO2010/075239A1 which describe various classes of agonists.

The S1PR1 receptor agonists are typically sphingosine analogs, such as 2-substituted 2-aminopropane-1,3-diol derivatives or 2-aminopropanol derivatives.

In one embodiment, the S1PR1 receptor agonists are compounds typically comprising a group of formula X

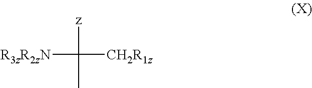

wherein Z is H, C₁-C₆ alkyl, C₂-C₆ alkenyl, C₂-C₆ alkynyl, phenyl, phenyl substituted with OH, C₁-C₆ alkyl substituted with 1 to 3 substituents selected from the group consisting of halogen, C₃-C₈ cycloalkyl, phenyl and phenyl substituted with OH; or CH₂—R_{4z} where R_{4z} is OH, acyloxy or a residue of formula (a)

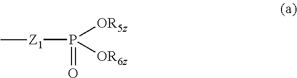

where Z₁ is a direct bond or O, preferably O;

each of R_{5z} and R_{6z}, independently, is H, or C₁-C₄ alkyl optionally substituted with 1, 2 or 3 halogen atoms;

R_{1z} is OH, acyloxy or a residue of formula (a); and each of R_{2z} and R_{3z}, independently, is H, C₁-C₄ alkyl, or acyl.

The group of formula (X) is a functional group which is attached as an end group to a group which may be hydrophilic or lipophilic and may comprise one or more aliphatic, alicyclic, aromatic and/or heterocyclic residues. The resulting molecule functions as an S1PR1 receptor agonist.

Preferably, at least one of Z and R_{1z} is or comprises a residue of formula (a).

Examples of S1PR1 receptor agonists comprise:

(i) the compounds described in EP627406A1, for example a compound of formula I:

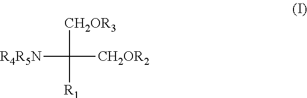

where R₁ is a linear or branched C₁₂-C₂₂ chain
which may have, in the chain, a bond or a heteroatom selected from a double bond, a triple bond, O, S, NR₆, where R₆ is H, C₁-C₄ alkyl, aryl(C₁-C₄)alkyl, acyl or (C₁-C₄ alkoxy)carbonyl, and carbonyl, and/or
which may have, as substituent, C₁-C₄ alkoxy, C₂-C₄ alkenyloxy, C₂-C₄ alkynyloxy, aryl(C₁-C₄)alkyloxy, acyl, C₁-C₄ alkylamino, C₁-C₄ alkylthio, acylamino, (C₁-C₄ alkoxy)carbonyl, (C₁-C₄ alkoxy)carbonylamino, acyloxy, (C₁-C₄ alkyl)carbamoyl, nitro, halogen, amino, hydroxyimino, hydroxyl or carboxy;

or R₁ is
a phenylalkyl where the alkyl is a linear or branched C₆-C₂₀ carbon-based chain; or
a phenylalkyl where the alkyl is a linear or branched C₁-C₃₀ carbon-based chain where said phenylalkyl is substituted with
   a linear or branched C₆⁻C₂₀ carbon-based chain optionally substituted with a halogen,
   a linear or branched C₆-C₂₀ alkoxy chain optionally substituted with a halogen,
   a linear or branched C₆-C₂₀ alkenyloxy chain,
   a phenyl(C₁-C₁₄)alkoxy, halophenyl(C₁-C₄)alkoxy, phenyl(C₁-C₁₄) alkoxy(C₁-C₁₄)alkyl, phenoxy(C₁-C₄)alkoxy or phenoxy(C₁-C₄)alkyl,
   a cycloalkylalkyl substituted with a C₆-C₂₀ alkyl,
   a heteroarylalkyl substituted with a C₆-C₂₀ alkyl,
   a heterocyclic C₆-C₂₀ alkyl, or
   a heterocyclic alkyl substituted with a C₆-C₂₀ alkyl,

and where the alkyl group may have:
in the carbon-based chain, a bond or a heteroatom, selected from a double bond, a triple bond, O, S, sulfinyl, sulfonyl, or NR₆, where R₆ is as defined previously, and
as substituent, C₁-C₄ alkoxy, C₂-C₄ alkenyloxy, C₂-C₄ alkynyloxy, aryl(C₁-C₄)alkyloxy, acyl, C₁-C₄ alkylamino, C₁-C₄ alkylthio, acylamino, (C₁-C₄ alkoxy)carbonyl, (C₁-C₄ alkoxy)carbonylamino, acyloxy, (C₁-C₄ alkyl)carbamoyl, nitro, halogen, amino, hydroxy or carboxy; and

each of R₂, R₃, R₄ and R₅, independently, is H, C₁-C₄ alkyl or acyl,

or a pharmaceutically acceptable salt or hydrate thereof;

(ii) the compounds described in WO 02/18395, for example, a compound of formula IIa or IIb

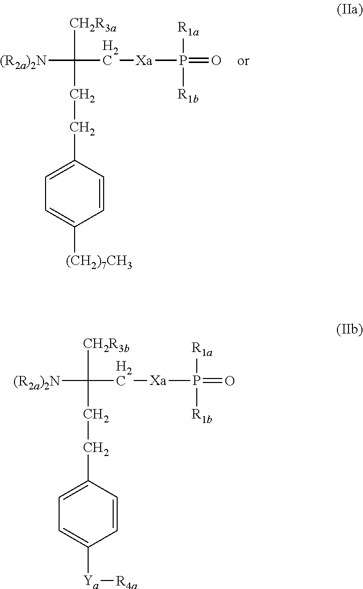

where Xₐ is O, S, NR₁ₛ or a —(CH₂)ₙₐ— group, said group being optionally substituted with 1 to 4 halogens; nₐ is 1 or 2, R₁ₛ is H or (C₁-C₄)alkyl, said alkyl being optionally substituted with a halogen, R₁ₐ is H, OH, (C₁-C₄)alkyl or O(C₁-C₄)alkyl, where the alkyl is optionally substituted with 1 to 3 halogens; R_{1b} is H, OH or (C₁-C₄)alkyl, where the alkyl is optionally substituted with a halogen; each R₂ₐ is independently selected from H or (C₁-C₄)alkyl, said alkyl being optionally substituted with a halogen; R₃ₐ is H, OH, halogen or O(C₁-C₄)alkyl where the alkyl is optionally substituted with a halogen; and R_{3b} is H, OH, halogen, (C₁-C₄)alkyl where the alkyl is optionally substituted with hydroxyl, or O(C₁-C₄)alkyl where the alkyl is optionally substituted with a halogen; Yₐ is —CH₂—, —C(O)—, —CH(OH)—, —C(═NOH)—, O or S, and R₄ₐ is (C₁-C₁₄)alkyl or (C₄-C₁₄)alkenyl;

or a pharmaceutically acceptable salt or hydrate thereof.

When the compounds of formula I, IIa or IIb have one or more asymmetric centers, the present invention should be understood to cover the various optical isomers, racemates, diastereoisomers and mixtures thereof. The compounds of formula IIa or IIb, when the carbon atom bearing the amine group is asymmetric, preferably have the R configuration for this carbon atom.

The compounds of formula I, IIa or IIb may be in free form or in salt form. Examples of pharmaceutically acceptable salts of the compounds of formula I, IIa or IIb comprise the salts of an inorganic acid, such as hydrochloride, hydrobromide and sulfate; the salts of an organic acid, such as acetate, fumarate, maleate, benzoate, citrate, malate, methanesulfonate and benzenesulfonate; or, when this is appropriate, the salts of metals, such as sodium, potassium, calcium, or aluminum; salts of amines, such as triethylamine; and salts of a dibasic amino acid, such as lysine. The compounds and salts thereof according to the invention also cover the hydrate and solvate forms thereof.

In the definitions above:
acyl may be an Ry-CO— residue where Ry is C₁-C₆ alkyl, C₃-C₆ cycloalkyl, phenyl or phenyl(C₁-C₄)alkyl,
unless otherwise indicated, alkyl, alkoxy, alkenyl or alkynyl may be linear or branched,
aryl may be phenyl or naphthyl, preferably phenyl,
“heterocyclic group” represents a heterocyclic group of 5 to 7 ring members, having 1 to 3 heteroatoms selected from S, O and N. Examples of such heterocyclic groups comprise the heteroaryl groups previously indicated, and the heterocyclic compounds corresponding to partially or completely hydrogenated heteroaryl groups, for instance furyl, thienyl, pyrrolyl, azepinyl, pyrazolyl, imidazolyl, oxazolyl, isoxazolyl, thiazolyl, isothiazolyl, 1,2,3-oxadiazolyl, triazolyl, tetrazolyl, thiadiazolyl, pyranyl, pyridyl, pyridazinyl, pyrimidinyl, pyrazinyl, tetrahydropyranyl, morpholinyl, thiomorpholinyl, pyrrolidinyl, pyrrolyl, imidazolidinyl, pyrazolidinyl, piperidinyl, piperazinyl, oxazolidinyl, isoxazolidinyl, thiazolidinyl or pyrazolidinyl. Preferred heterocyclic groups are the heteroaryl groups with 5 or 6 ring members, and the most preferred are morpholinyl, thiomorpholinyl or piperidinyl groups.

When the carbon-based chain as R₁ is substituted in the compounds of formula I, this chain is preferably substituted with a halogen, nitro, amino, hydroxyl or carboxy. When the carbon chain is interrupted with an optionally substituted phenylene, the carbon-based chain is preferably unsubstituted. When the phenylene group is substituted, it is preferably substituted with a halogen, nitro, amino, methoxy, hydroxyl or carboxy.

Preferred compounds of formula I are those wherein R₁ is C₁₃-C₂₀ alkyl, optionally substituted with nitro, halogen, amino, hydroxyl or carboxy, and, more preferentially, those where R₁ is a phenylalkyl substituted with a C₆-C₁₄ alkyl chain optionally substituted with a halogen and the alkyl group is a C₁-C₆ alkyl optionally substituted with a hydroxyl. More preferentially, R₁ is a phenyl(C₁-C₆)alkyl substituted on the phenyl with a linear or branched, preferably linear, C₆-C₁₄ alkyl chain. The C₆-C₁₄ alkyl chain may be ortho, meta or para, preferably para.

Preferably, each of R₂ to R₅ is H.

A preferred compound of formula I is 2-amino-2-tetradecyl-1,3-propanediol.

A particularly preferred compound of formula I is FTY720, i.e. 2-amino-2-[2-(4-octylphenyl)ethyl]propane-1,3-diol in its free form or in the form of one of its pharmaceutically acceptable salts, or a prodrug thereof.

In one embodiment, the compound according to the invention is FTY720 hydrochloride, as indicated hereinafter:

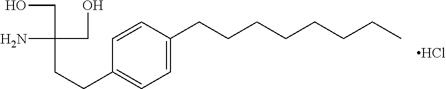 [SMILES: CCCCCCCCc1ccc(CCC(N)(CO)CO)cc1 ; Cl]

A preferred compound of formula IIa is FTY720-phosphate or FTY720-P (R₂ₐ is H, R₃ₐ is OH, Xₐ is O, R₁ₐ and R_{1b} are OH).

A preferred compound of formula IIb is a compound-phosphate wherein R₂ₐ is H, R_{3b} is OH, Xₐ is O, R₁ₐ and R_{1b} are OH, Yₐ is O and R₄ₐ is heptyl.

According to one embodiment, the compound according to the invention is selected from the group comprising sphingosine-1-phosphate, FTY720, FTY720-P, AUY954, CYM-5442, CYM-5181, SEW2871, VPC01091, DS-SG-44, KRP-203-P, DihydroS1P, Compound 26, Compound 12, sphingosylphosphorylcholine and AFD-R. These compounds are in particular mentioned by Dong-Soon IM in Acta Pharmacologica Sinica (2010) 31:1213-1222.

The term “Compound 26” is intended to mean the compound described in the publication Li et al., “Discovery of potent 3,5-diphenyl-1,2,4-oxadiazole sphingosine-1-phosphate (S1P₁) receptor agonists with exceptional selectivity against S1P₂ and S1P₃”, Journal of Medicinal Chemistry, 2005 and having the following formula:

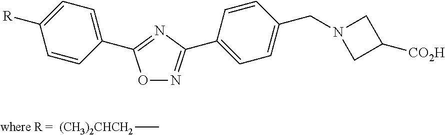

The term “Compound 12”, is intended to mean the compound described in the publication Hanessian et al., “Constrained azacyclic analogues of the immunomodulatory agent FTY720 as molecular probes for sphingosine 1-phosphate receptors”, Bioorganic & Medicinal Chemistry Letters, 2007 and having the following formula:

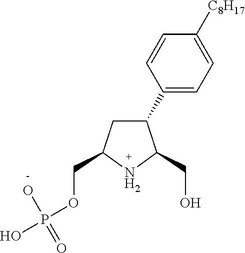

The term “AFD-R”, is intended to mean the compound described in the publication Brinkmann et al., “The immune modulator FTY720 targets sphingosine-1-phosphate receptors”, The Journal of Biological Chemistry, 2002 and having the following formula:

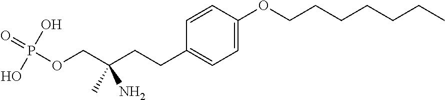 [SMILES: CCCCCCCOc1ccc(CC[C@@](C)(N)COP(=O)(O)O)cc1]

Preferably, the compound according to the invention is an S1PR1, S1PR3, SPR4 and S1PR5 receptor agonist. More preferably, the compound according to the invention is FTY720 (also known as “fingolimod”) or FTY720-P, which is the phosphorylated form of FTY720. FTY720 and FTY720-P have in particular been described by Mandala S. et al. in Science 2002; 296:346-9 and by Brinkmann V. et al. in J Biol Chem 2002; 277:21453-7. FTY720 is a substrate of sphingosine kinase 2 and is, in its phosphorylated form, an S1PR1, S1PR3, S1PR4 and S1PR5 receptor agonist.

FTY720-P is particularly relevant for use in the treatment of an HIV infection. This is in particular explained by the synergy of the effects of FTY720-P on the S1PR1, S1PR3, S1PR4 and S1PR5 receptors. The inventors have shown the protective effect against HIV infection of this compound on cell lines and primary cells. Finally, they have also shown the effect of this molecule in vivo on mice grafted with human lymphocytes before infection with HIV.

In addition, the inventors have shown that FTY720-P makes it possible to obtain a decrease in HIV infection in cells not expressing S1PR1. This result confirms that the S1PR3 and/or S1PR4 and/or SPR5 receptors also have an essential role in protection against HIV infection.

In one embodiment, the agonists of the invention are agonists specific for the S1PR1 receptor and for at least one receptor selected from S1PR2, S1PR3, S1PR4, and S1PR5.

In another particular embodiment, the S1PR1 receptor agonist is specific for the S1PR1 receptor. Typically, said compound is selected from CYM-5442, CYM-5181 and SEW2871. Even more preferably, this compound is SEW2871. An agonist specific for the S1PR1 receptor is typically a compound which has an at least 20 times greater, typically 100, 500, 1000 or 2000 times greater, specificity for the S1PR1 receptor, compared with another S1PR receptor (e.g. S1PR2-5), as measured by the ratio of the EC₅₀ for the S1PR1 receptor/EC₅₀ for the other S1PR receptor (e.g. S1PR2-5) as measured by a ³⁵S-GTPγS binding assay, and where said compound has an EC₅₀ for the S1PR1 receptor of 100 nM or less, as measured by the ³⁵S-GTPγS binding assay. The ³⁵S-GTPγS binding assay is described in patent application WO03/097028 and follows the protocol below:

The ³⁵S-GTPγS binding assay is carried out as described by D S. Im et al., Mol. Pharmacol. 2000; 57-753. The binding between the GTPγS and the G proteins, mediated by the ligand, is measured in a GTP binding buffer (in mM: 50 HEPES; 100 NaCl, 10 MgCl₂, pH 7.5) using 25 μg of a membrane prepared from transiently transfected HEK293 cells. The ligand is added to the membranes in the presence of 10 μM of GDP and of 0.1 nM of ³⁵S-GTPγS (1200 Ci/mmol) and incubated at 30° C. for 30 minutes. The bound GTPγS is separated from the unbound GTPγS using a Brandel collector (Gaithersburg, Md.), and counted with a liquid scintillant counter.

Treatment Methods

The invention also relates to methods for treating a subject suffering from an HIV infection comprising the step of administering to said subject, a therapeutically effective amount of at least one compound which is an agonist of at least one receptor, selected from S1PR1, S1PR2, S1PR3, S1PR4 and S1PR5 receptors, and preferentially which is an S1PR1 receptor agonist.

The compounds according to the invention can also be administered in the form of pharmaceutical compositions, as defined hereinafter.

The term “therapeutically effective amount” is intended to mean an amount sufficient for treating and/or preventing HIV infection.

Pharmaceutical Compositions

The compounds according to the invention can also be used for preparing pharmaceutical compositions for the treatment of HIV infections.

Thus, the invention also relates to pharmaceutical compositions for use in methods for treating HIV infections in humans or animals, said compositions comprising at least one compound according to the invention and a pharmaceutically acceptable vehicle.

Any compound according to the invention can be combined with any type of pharmaceutically acceptable vehicle or excipient, and optionally with a sustained diffusion matrix, such as a biodegradable polymer, for forming a pharmaceutical composition according to the invention.

The term “pharmaceutically acceptable” refers to molecular entities and compositions which do not produce an opposite or allergic reaction or other unwanted reaction when they are administered to a mammal, particularly a human being. A pharmaceutically acceptable vehicle or excipient may be solid, semi-solid or liquid.

The form of the pharmaceutical compositions, their route of administration, their dosage and their posology naturally depend on the severity of the infection, on its stage of progression, on the age, sex and weight of the subject to be treated, etc.

The pharmaceutical compositions according to the invention can be formulated for topical, oral, intranasal, parenteral, intravenous, intramuscular, or subcutaneous administration, or the like.

According to one embodiment of the invention, said HIV infection is an HIV-1 infection. According to another embodiment, said HIV infection is an HIV-2 infection.

The following examples are given solely by way of illustration, and cannot limit the scope of the invention.

### FIGURE LEGENDS

FIG. 1: Measurement of the infectability of the HOS CCR5 LacZ and HOS CCR5 S1PR1 lines with the Ad8Luc virus

The infectability is measured via luciferase activity. This figure shows that the presence of the S1PR1 receptor inhibits the infection of the HOS line by the HIV virus.

FIG. 2: Effect of FTY720-P on an HOS CCR5 S1PR1 cell line

The infectability is measured via luciferase activity. This figure shows that pre-incubation with FTY720-P of the cells expressing S1PR1 decreases the infection.

FIG. 3: Measurement of the infectability on an HOS CCR5 line not expressing S1PR1

HOS CCR5 lacZ cells were pre-incubated for 1 hour with:
an S1PR1 specific agonist, SEW2871, or
the natural agonist of all the S1PR receptors (S1PR1 to S1PR5), sphingosine-1-phosphate (S1P), or
an agonist of S1PR1, S1PR3, S1PR4 and S1PR5, FTY720-P.

No significant difference is observed with SEW2871, whereas S1P and FTY720-P make it possible to obtain a decrease of approximately 50% of the HIV infection.

FIG. 4: Inhibition of the infection of primary lymphocyte cells by FTY720-P

This figure shows the effect of S1PR1 and FTY720-P on peripheral blood mononuclear cells (PBMCs).

This figure confirms the effect of FTY720-P on primary cells.

FIG. 5: Measurement of viral production in the culture medium of primary cells infected with an Ad8 replicative virus and pre-incubated with FTY720-P

This figure shows a clear inhibition of viral production by the PBMCs.

FIG. 6: Effect of FTY720 in vivo

This figure shows a clear decrease in viremia in mice grafted with human lymphocyte cells and infected with HIV. This figure confirms the in vivo effect of FTY720 on the prevention of HIV infection.

### EXAMPLES

Materials and Methods

The materials and methods used for all of the experimental section are detailed hereinafter.

Cells

The HOS-CD4 line (AIDS Reagent Program, Rockville, Md.) and the HEK-293T line (simian virus 40 T antigen-transformed human embryonic kidney 293T, Genethon) are cultured in DMEM medium supplemented with 10% of FCS (fetal calf serum), 10 mM of glutamax-1, 100 U/ml of penicillin and 100 μg/ml of streptomycin. The HEK-293T CD4+ CCR5+ line (gift from Martine Biard-Piechaczyk, Institute of Biology, Montpellier, France) was obtained by transfection of CD4 and CCR5 genes using the HEK-293T line. The human PBMCs in culture are isolated from blood of healthy donors by density centrifugation on lymphocyte separation medium (Eurobio) and cultured in RPMI medium supplemented like the DMEM at 37° C., 5% CO₂.

Effect of S1PR1 on the Infection of the HEK-293T Line

In order to test the influence of the expression of S1PR1 on infection with HIV-1, 50 000 HEK-293T CD4+ CCR5+ cells were transiently transfected by lipofection, by incubating them for 24 hours with a mixture of 50 ng of CMV-S1PR1 plasmid, 20 ng of the pRL-TK-Renilla Luciferase plasmid (Promega) and 0.5 μl of lipofectamine (Lipofectamine™ 2000, Invitrogen) in 150 μl of DMEM medium, 10% FCS, in a 96-well plate, incubated beforehand with a solution of D-polylysine for 30 min. The plasmid clone of S1PR1 was obtained from Missouri S&T cDNA Resource Center (www.cdna.org). A fragment bearing S1PR1 was cloned under the control of the CMV promoter, into the pcDNA3.1+ expression vector (Invitrogen). 24 h after the transfection, the wells are washed with DMEM (BioWhittaker), and then the cells are infected with 40 ng of the Ad8-luc virion in 180 μl of culture medium. The Ad8-luc virion is a nonreplicative R5-pseudotyped virus obtained by cotransfection of HEK293T cells with the pNL4.3 Luc.R-E-transfer plasmid which bears a defective env viral gene and also the Firefly luciferase gene inserted into the nef viral gene (AIDS Reagent Program), and the pCMV-AD8-env plasmid which encodes the R5 envelope of the HIV-1 prototype Ad8 (Cho, Shibata & Martin (1996) J. Virol. 70, 7318-7321). 24 h after the infection, the cells are washed once with culture medium, and put back in culture with 200 μl of DMEM, 10% FCS. 40 h to 48 h after the infection, the cells are washed once with PBS, and lysed with 50 μl of buffer, and then the Firefly and Renilla luciferase activities are measured sequentially in a luminometer using the Dual-Luciferase® Reporter Assay system (Promega, cat: E1910). The Renilla luciferase activity is used as a measure of the level of cell metabolism.

Transduction of HOS Cells for the Expression of CCR5 and S1PR1

In order to produce HIV vectors expressing the CCR5, lacZ and S1PR1 genes, the pWPXL-CCR5 plasmids (Desmetz et al., 2007, Clin Immunol 123, 148-154), pHRCMV-lacZ (Naldini et al., 1996, Science 272, 263-267) and pWPXL-S1PR1 plasmids were cotransfected with the p8.2 packaging plasmid and the pMD2G plasmid which encodes the envelope of the vesicular stomatitis virus, into 293T cells as described previously (Lin et al., 2002, PNAS 99, 15590-15595). In order to construct the pWPXL-S1PR1 plasmid, the coding sequence of S1PR1 was amplified by PCR from the pCDNA3.1-S1PR1 plasmid, and then the BamH1-Spe1 fragment obtained was cloned into the pWPXL lintiviral vector (addgene.org). The HOS cells were transduced (Lin et al., 2002) first with the HIV-CCR5 vector, and the line obtained was retransduced with the HIV-S1PR1 vector or the HIV-lacZ vector with equal amount of virus in terms of p24 equivalent. The membrane expression of the CCR5 and S1PR1 receptors was evaluated by flow cytometry (FACScalibur, BD Biosciences) after labeling of the cells with the anti-human CD195 (BD Pharmingen) and anti-human EDG1 (R&D) mouse monoclonal antibodies, respectively.

Relating to Infection of HOS Cells with the Nonreplicative Ad8-luc Virus

50 000 HOS cells are cultured in triplicate in a 96-well plate, and then infected with 50 ng of Ad8-luc virus. 24 h after the infection, the cells are washed twice with PBS, and put back in culture for 48 h. The cells are then washed once with PBS, and lysed with 50 μl of buffer, and then the Firefly luciferase activity is measured in a luminometer using the Promega kit (Luciferase Assay System).

Tests Relating to Infection of PBMCs with the Replicative Ad8 Virus

The PBMCs are activated by incubation for 72 h in culture medium supplemented with phytohemagglutinin (PHA, 1 μg/ml) and with interleukin 2 (IL2, 100 U/ml), and then, after washing with RPMI, incubated in 96-well plates in triplicate at 200 000 cells in 200 μl per well. They are then infected with 70 ng of p24 equivalent of the HIV-1 prototype Ad8 for 18 h, and then washed twice with PBS and incubated for 11 days, while adjusting the number of cells at each sampling of the culture supernatant on D4, D7 and D9. The virus production is monitored in the culture supernatant by measuring the concentration of the gag p24 protein by ELISA using a commercial kit (Innotest HIV AG MAB, Ingen, ref:80563).

Pharmacological Molecule Tests

For the HEK-293T CD4+ CCR5+ cells, 24 h after infection with the S1PR1 plasmid or with the empty vector, the cells are incubated for 30 min and then infected with the anti-CCR5 antibody at 10 μg/ml (clone 2D7, Pharmingen, ref. 555991), or with MIP-1β at 100 ng/ml (R&D systems, ref. 271-BME) or with (S)-FTY720 Phosphate (FTY720-P) at 80 ng/ml (Echelon, ref. B-0721).

For the HOS cells and the PBMCs, the S1P (Enzo, sold by covalab, ref. SL-140), FTY720-P (Echelon) or SEW2871 (Cayman, ref. 10006440) molecules were added to the culture medium 1 hour before the infection at the concentrations indicated.

Immunologically Humanized Mouse Animal Model

SCID mice have the cb17/Icr-Prkdc^{scid}/Crl genotype. These immunodepressed mice are housed in an A3/L3 animal house in cages with a filter lid, in a ventilated cage rack. After one week of acclimatization, the animals are reconstituted by intraperitoneal injection of 30×10⁶ PBMCs obtained by density centrifugation of the leukocytes of an uncertified cytapheresis annulus from a healthy volunteer donor. The reconstitution is evaluated on the 13th day by assaying the human immunoglobulins (Igs) present in the serum of the mice by means of an Elisa assay using the anti-human whole Ig-PO antibody (MP Biomedical, cat n° 55230). The mice whose total Ig concentration is greater than 100 μg/ml are kept for the experiment, and are infected 14 days after the reconstitution with the HIV-1 strain JR-CSF (R5 strain) at 1000 TCID50 in 100 μl. Daily gavage with 100 μl of FTY720 (Cayman, sold by Interchim ref. BM8045) dissolved at 60 μg/ml in distilled water is begun one day before the infection and continued for 12 days. The untreated mice are given the same volume of distilled water by gavage. A 50 μl blood sample is taken retro-orbitally and diluted with 1 ml of human plasma in order to assay the viral load on the 6th, 9th and 12th day after the infection, after anesthesia with isoflurane. The viremies are evaluated by quantification of the viral ARN in the plasma of the mice by qPCR with the AmpliPrep/COBAS TaqMan HIV-1 test (Roche Diagnostics, ref. 05212294190). The values below 20 copies of RNA/ml (i.e. 400 copies/ml of mouse serum) which are below the detection threshold of the kit are considered to be equal to zero.

TR-FRET Experiments

The coding sequence of S1PR1 was amplified by PCR starting from the 2nd codon up to the stop codon between Mlu1 and Xba1 sites, and then inserted, using these restriction sites, into the pRK5-HA-SNAP-mGlu2 and pRK5-FLAG-CLIP-mGlu2 plasmids (Doumazane, E. et al., FASEB J. 2010 Sep. 27) as a replacement for the mGlu2 gene, in such a way that the S1PR1 receptor is fused, at the N-terminal end, either with a hemagglutinin (HA) epitope and the SNAP enzyme, or with a FLAG epitope and the CLIP enzyme, inserted behind a signal peptide.

HEK293 cells were transfected by lipofection with DNA of the CLIP-CCR5 plasmid (7 ng per 100 000 cells) and of the SNAP-RCPG plasmid (range of from 1 to 100 ng per 100 000 cells) and deposited, at a rate of 100 000 cells per well, black 96-well plates pre-incubated with a polyornithine solution. 24 to 30 h after the transfection, the adherent cells are washed and then incubated with culture medium containing the substrates of the SNAP and CLIP enzymes coupled to fluorophores, for 2 h at 37°. The fluorophores are Lumi4®-Terbium Cryptate bonded to O⁶-benzylguanine (BG-Lumi4) used at 0.3 μM, and fluorescein bonded to O²-benzylcytosine (BC-Fluorescein) used at 1 μM, which are substrates of the SNAP and CLIP enzymes, respectively. BG-Lumi4 and BC-Fluorescein come from Cisbio Bioessays (Bagnols-sur-Cèze, France). Under these conditions, the labeling of the receptors with the fluorophore is specific for SNAP or CLIP, total (100% of the receptors are labeled) and restricted to the receptors present at the cell surface (Doumazane, E. et al., FASEB J. 2010 Sep. 27). Furthermore, the Lumi4 and fluorescein fluorescence intensities are proportional to the number of receptors present at the cell surface (Doumazane, E. et al., FASEB J. 2010 Sep. 27). The cells are then washed 4 times with Tris-Krebs buffer at 37° C. (Maurel, D. et al., 2008, Nat Methods 5:561-567). The fluorescence and the TR-FRET are measured in 100 μl of Tris-Krebs buffer in an Infinite F500 spectrofluorimeter (Tecan, Männedorf, Switzerland) with the following parameters: Lumi4 (excitation at 320 nm, emission at 620 nm, 150 μs delay and 500 μs integration time), fluorescein (excitation at 485 nm, emission at 520 nm, 0 μs delay and 1000 μs integration time), trFRET (excitation at 320 nm, emission at 520 nm, 150 μs delay and 500 μs integration time). The FRET signal is corrected by subtraction of the nonspecific FRET obtained after transfection of the SNAP receptor alone, and of the nonspecific FRET obtained with the BG-Lumi4 substrate alone. The Lumi4 and fluorescein fluorescence values are corrected by subtraction of the nonspecific signal measured after transfection of an empty plasmid.

Results

I. Effect of the Agonists of the Invention on HEK293T Lines Expressing the CD4 CCR5 Receptors

Study of the Capacity of S1PR1 to Interfere with HIV Infection

The gene encoding S1PR1 was cloned into an expression vector under the control of the CMV promoter. An HEK293T line expressing the CD4 CCR5 receptors constitutively and at a sufficient level to allow infection was used.

The results show that, when a plasmid which allows expression of the S1PR1 receptor at the membrane in this line is introduced, the level of infection is reduced by a factor of approximately two compared with the control (empty plasmid). As expected, the provision of a CCR5 plasmid which increases the membrane level of the coreceptor of HIV gave a level of infection greater than the control. In these experiments, it is verified that the effect on the infection, of the S1PR1 and CCR5 plasmids, is not the result of an overall effect on cell metabolism.

Effect, on the Infection, of the Stimulation of the S1PR1 Receptor by the FTY720-P Agonist

Once the capacity of S1PR1 to interfere with HIV infection had been demonstrated, the effect of an S1PR1 agonist on HIV infection was tested, using the same method, but including a step of incubating the cells with the test agonist just before the infection.

The results show that, in the presence of the unstimulated S1PR1 receptor, the infection is decreased by 60% compared with the empty plasmid control and that, in the presence of the S1PR1 receptor stimulated by the FTY720-P agonist for 30 min before the addition of the virus, the infection is then reduced by 90% compared with the empty plasmid control. This decrease in infection with FTY720-P is of the same level as that which is obtained in this experiment when the cells are incubated under the same conditions with an anti-CCR5 antibody or with a CCR5 ligand (MIP1-β), which are known to inhibit HIV infection (Wu, L. et al., 1997, J Exp Med 186:1373-1381; Cocchi, F. et al., 1995, Science 270 (5243):1811-1815).

Demonstration of a Heterodimerization of S1PR1 with CCR5 Using the HTRF Technique

GPCRs can act as homodimers, but also via heterodimeric interaction. In order to specify the mechanism by which S1PR1 inhibits infection, it was tested for its capacity to heterodimerize with CCR5. For this, a TR-FRET (Time Resolved-Fluorescence Resonance Energy Transfer) technology, HTRF (http://www.htrf.com/technology/) was used. In this technology, which allies the principles of TRF (Time-Resolved Fluorescence) and of FRET (Fluorescence Resonance Energy Transfer), each of the CCR5 and S1PR1 receptors is expressed at the membrane surface as a translational fusion with the SNAP and CLIP enzymes, respectively, which enable the specific and irreversible binding of two fluorophores coupled to substrates of these enzymes. A FRET signal is detected when the 2 fluorophores are at distances compatible with heterodimerization between the receptors.

The results show that the FRET signal obtained between the S1PR1 and CCR5 receptors is comparable to that obtained for the CCR5 homodimer, which attests to a direct interaction at the membrane between CCR5 and S1PR1, probably within a heterodimeric complex.

II. Effect of the Agonists of the Invention on HOS-CD4 Lines

The inventors corroborated their first results obtained on HEK293T lines expressing CD4 CCR5 receptors using HOS-CD4 cells.

S1PR1 Expression Inhibits the Infection of an HOS Line by HIV

An HOS (Human OsteoSarcoma) line expressing the CD4 receptor was transduced with a lentiviral vector enabling the constitutive expression of the CCR5 coreceptor at the plasma membrane in order to make it infectable by the HIV-1 virus. This HOS CCR5 line is transduced again so as to obtain expression at the membrane of the S1PR1 receptor (HOS CCR5 S1PR1). In parallel, a line transduced with the lacZ gene (HOS CCR5 lacZ) is obtained by the same technique as infectability control.

These two lines are infected with various doses of Ad8-luc virus and their infectability is evaluated for 72 h after the infection by measuring luciferase activity (FIG. 1).

It is therefore noted that the simple presence of the S1PR1 receptor inhibits the infection of the HOS line by HIV. This effect is very marked, if the fact that it is a one-round virus, which therefore effects only one replication cycle, is taken into consideration. This result confirms the results previously presented on the HEK293 line, where the virus was used at a single dose.

FTY720-P Accentuates the Inhibition of Infection of the HOS CCR5 S1PR1 Line

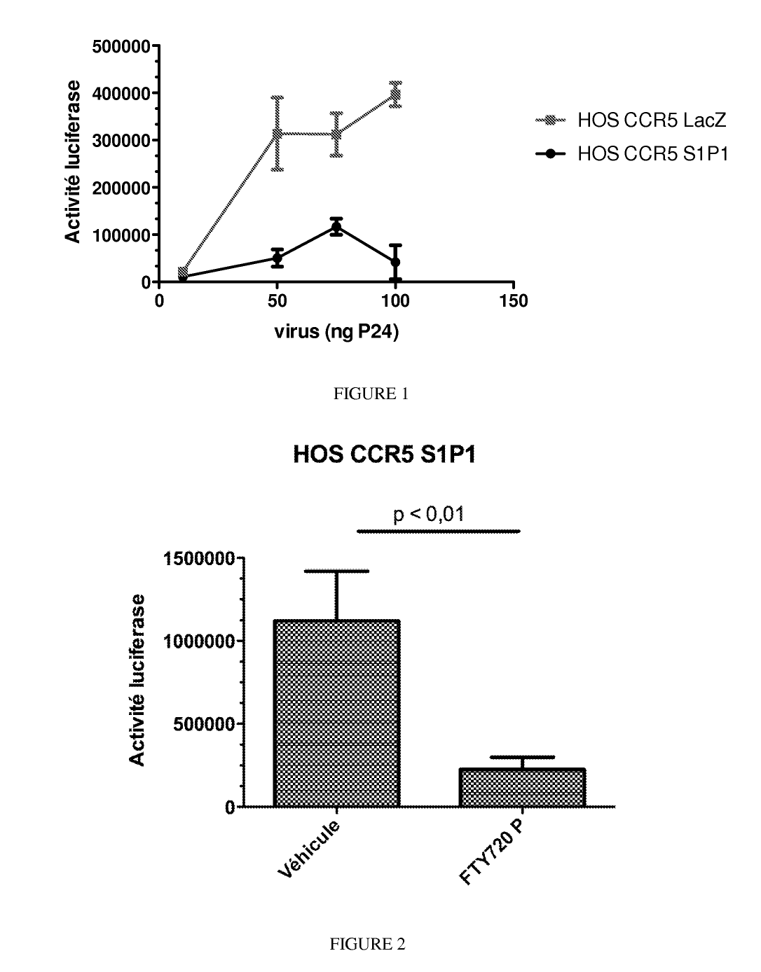

HOS CCR5 S1PR1 cells are exposed for one hour to FTY720-P (33 μM) and then infected as previously (FIG. 2).

In this experiment, the preincubation, with FTY720-P, of the cells expressing the S1PR1 receptor reduced the infection by a further 80%, in addition to the inhibition noted in the presence of unstimulated S1PR1.

FTY720-P Inhibits the Infection of the HOS CCR5 Line in the Absence of S1PR1

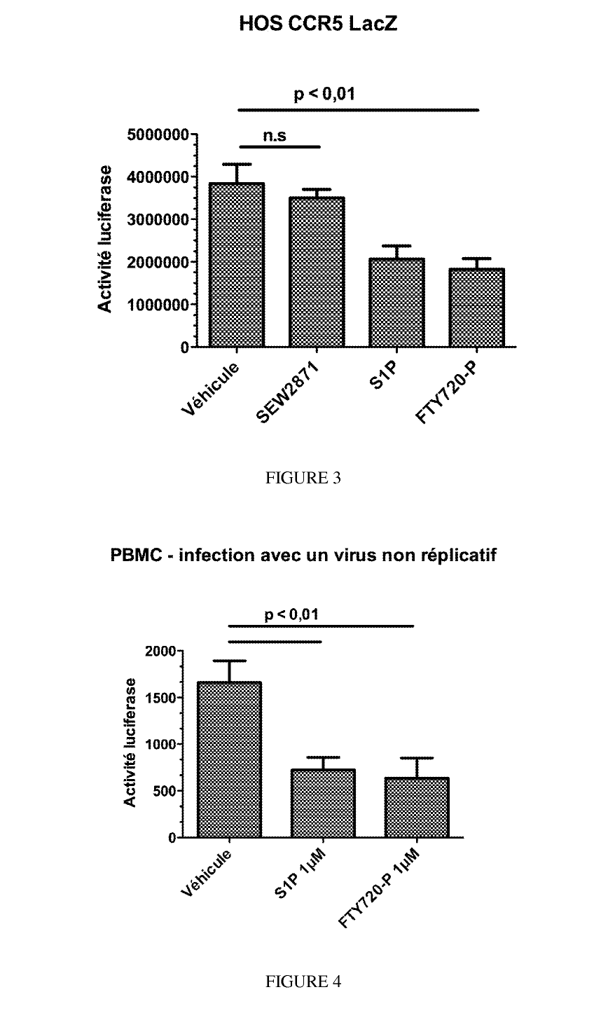

In this experiment, the inventors tested the effect of various S1PR receptor agonists on infection, by the Ad8-luc strain, of the HOS CCR5 line which does not express S1PR1. For this, HOS CCR5 lacZ cells were preincubated for one hour, either with an S1PR1 specific agonist, SEW2871 (33 μM), or with the natural agonist of all S1PR receptors (S1PR1 to S1PR5), sphingosine-1-phosphate (S1P, 33 μM), or with FTY720-P (33 μM) which is an S1PR1, S1PR3, S1PR4 and S1PR5 agonist (FIG. 3).

It appears first that SEW2871 does not significantly modify the infection in this experiment, which is an expected result given that this line does not express S1PR1.

On the other hand, S1P and FTY720 decrease the infection measured by approximately 50%, very significantly (student's test, p<0.01). It therefore proves to be the case that, in addition to the fact that the stimulation of S1PR1 decreases the infection of a line which expresses this receptor at its surface, agonists of S1PR2 to S1PR5 receptors also have a protective effect against infection.

III. Inhibition of the Infection of Primary Lymphocyte Cells by FTY720-P

Peripheral blood mononuclear cells (PBMCs) isolated from a healthy donor were preincubated in the presence of FTY720-P at 1 μM or of S1P at 1 μM, and then infected with Ad8-luc virus for 72 h, before measuring the luciferase activity. The results show an inhibition of approximately 60% of the infection by these two agonists (FIG. 4).

Expression of the S1PR1 receptor at the surface of the PBMCs was not detected by flow cytometry (despite the presence of mRNA in these cells).

It is therefore probable that the effect of these agonists is explained by the stimulation of at least one other receptor among S1PR2, S1PR3, S1PR4 and S1PR5.

This result confirms the fact that the inhibition of infection by FTY720-P is not only due to its effect on S1PR1.

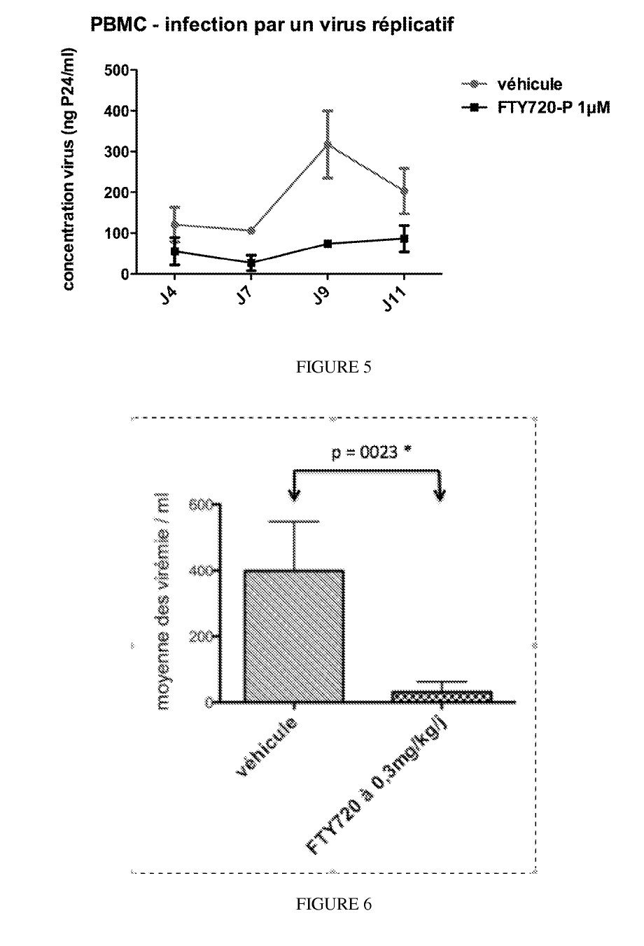

In order to confirm this effect on primary cells, PBMCs from healthy donors were preincubated with FTY720-P at 1 μM for one hour, and then infected with replicative Ad8 virus. The amount of virus produced in the culture medium was evaluated by measuring the p24 viral protein at 4, 7, 9 and 11 days after the infection (FIG. 5).

A clear inhibition of viral production by the PBMCs, which reaches 77% on average on the 9th day, is observed.

Thus, the inventors demonstrated the effect of the sphingosine-1-phosphate receptor agonists both on cell lines and on primary cells.

IV. Test of the Efficacy of the Anti-HIV Activity of FTY720 in vivo

This experiment makes it possible to confirm, in vivo, in the model of humanized SCID mice (hu-PBL-SCID) infected with the HIV-1, the anti-HIV activity of FTY720 shown previously in vitro on lines or primary cells.

Two groups of animals were compared with respect to HIV infection; a control group of 8 untreated animals and a group of 6 animals treated with FTY720. The experiment was carried out as follows:
D-21: Reception of the mice in an A3 animal house and adaptation to this environment for one week.
D-14: Immune reconstitution of the animals by intraperitoneal injection of human PBMCs.
D-1: Evaluation of the immune graft and constitution of two groups of mice having a comparable reconstituted mouse distribution. Beginning of the treatment. Gavage with FTY720 at 0.3 mg per kg and per day or with the vehicle, then daily gavage for 15 days.
D0: Infection of the mice with the HIV-1 virus type R5.
D6, 9, 12: Assaying of the viral load and sacrifice of the animals on D12.

The average value of the viremias for each of the 2 groups is represented in FIG. 6 (*: student's test, Welch correction). A significant decrease (p=0.023) in the infection of the treated animals is observed. This experiment therefore confirms the anti-viral effect of FTY720 on HIV in vivo, in the “humanized” mouse model.

1. A compound which is an agonist of at least one receptor selected from S1PR1, S1PR2, S1PR3, S1PR4 and S1PR5 receptors, for use in a method for treating an HIV infection in humans or animals.

2. The compound as claimed in claim 1, characterized in that said agonist is an S1PR1 receptor agonist.

3. The compound as claimed in either one of claims 1 and 2, characterized in that said agonist is also an agonist of at least one receptor selected from S1PR2, S1PR3, S1PR4 and S1PR5.

4. The compound as claimed in either one of claims 1 and 2, said compound being selected from the group comprising sphingosine-1-phosphate, FTY720, FTY720-P, AUY954, CYM-5442, CYM-5181, SEW2871, VPC01091, DS-SG-44, KRP-203-P, DihydroS1P, Compound 26, Compound 12, sphingosylphosphorylcholine and AFD-R.

5. The compound as claimed in any one of claims 1-4, said compound being FTY720 or FTY720-P.

6. A pharmaceutical composition for use in a method for treating an HIV infection in humans or animals, said composition comprising a compound as defined in any one of claims 1-5 and a pharmaceutically acceptable vehicle.

7. The compound as claimed in any one of claims 1-5, or the composition as claimed in claim 6, said HIV infection being an HIV-1 infection.

8. The compound as claimed in any one of claims 1-5, or the composition as claimed in claim 4, said HIV infection being an HIV-2 infection.


SEQUENCE LISTING (6 sequences)

SEQ ID NO: 1 - protein, 382 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 2 - protein, 353 aa, Homo sapiens
MGSLYSEYLNPNKVQEHYNYTKETLETQETTSRQVASAFIVILCCAIVVENLLVLIAVARNSKFHSAMYLFLGNLAASDLLAGVAFVANTLLSGSVTLRLTPVQWFAREGSAFITLSASVFSLLAIAIERHVAIAKVKLYGSDKSCRMLLLIGASWLISLVLGGLPILGWNCLGHLEACSTVLPLYAKHYVLCVVTIFSIILLAIVALYVRIYCVVRSSHADMAAPQTLALLKTVTIVLGVFIVCWLPAFSILLLDYACPVHSCPILYKAHYFFAVSTLNSLLNPVIYTWRSRDLRREVLRPLQCWRPGVGVQGRRRGGTPGHHLLPLRSSSSLERGMHMPTSPTFLEGNTVV

SEQ ID NO: 3 - protein, 378 aa, Homo sapiens
MATALPPRLQPVRGNETLREHYQYVGKLAGRLKEASEGSTLTTVLFLVICSFIVLENLMVLIAIWKNNKFHNRMYFFIGNLALCDLLAGIAYKVNILMSGKKTFSLSPTVWFLREGSMFVALGASTCSLLAIAIERHLTMIKMRPYDANKRHRVFLLIGMCWLIAFTLGALPILGWNCLHNLPDCSTILPLYSKKYIAFCISIFTAILVTIVILYARIYFLVKSSSRKVANHNNSERSMALLRTVVIVVSVFIACWSPLFILFLIDVACRVQACPILFKAQWFIVLAVLNSAMNPVIYTLASKEMRRAFFRLVCNCLVRGRGARASPIQPALDPSRSKSSSSNNSSHSPKVKEDLPHTAPSSCIMDKNAALQNGIFCN

SEQ ID NO: 4 - protein, 384 aa, Homo sapiens
MNATGTPVAPESCQQLAAGGHSRLIVLHYNHSGRLAGRGGPEDGGLGALRGLSVAASCLVVLENLLVLAAITSHMRSRRWVYYCLVNITLSDLLTGAAYLANVLLSGARTFRLAPAQWFLREGLLFTALAASTFSLLFTAGERFATMVRPVAESGATKTSRVYGFIGLCWLLAALLGMLPLLGWNCLCAFDRCSSLLPLYSKRYILFCLVIFAGVLATIMGLYGAIFRLVQASGQKAPRPAARRKARRLLKTVLMILLAFLVCWGPLFGLLLADVFGSNLWAQEYLRGMDWILALAVLNSAVNPIIYSFRSREVCRAVLSFLCCGCLRLGMRGPGDCLARAVEAHSGASTTDSSLRPRDSFRGSRSLSFRMREPLSSISSVRSI

SEQ ID NO: 5 - protein, 310 aa, Homo sapiens
MESGLLRPAPVSEVIVLHYNYTGKLRGARYQPGAGLRADAVVCLAVCAFIVLENLAVLLVLGRHPRFHAPMFLLLGSLTLSDLLAGAAYAANILLSGPLTLKLSPALWFAREGGVFVALTASVLSLLAIALERSLTMARRGPAPVSSRGRTLAMAAAAWGVSLLLGLLPALGWNCLGRLDACSTVLPLYAKAYVLFCVLAFVGILAAICALYALAGLAAHAQRGAPGLCGMLGPPHPAAVARRGVPGAHLSCTPAGRSLPGTGHGQLTSEPHHLHAHQPRPAPRAPAPGLLRTPLLRQRPEWLPAVGERG

SEQ ID NO: 6 - protein, 398 aa, Homo sapiens
MESGLLRPAPVSEVIVLHYNYTGKLRGARYQPGAGLRADAVVCLAVCAFIVLENLAVLLVLGRHPRFHAPMFLLLGSLTLSDLLAGAAYAANILLSGPLTLKLSPALWFAREGGVFVALTASVLSLLAIALERSLTMARRGPAPVSSRGRTLAMAAAAWGVSLLLGLLPALGWNCLGRLDACSTVLPLYAKAYVLFCVLAFVGILAAICALYARIYCQVRANARRLPARPGTAGTTSTRARRKPRSLALLRTLSVVLLAFVACWGPLFLLLLLDVACPARTCPVLLQADPFLGLAMANSLLNPIIYTLTNRDLRHALLRLVCCGRHSCGRDPSGSQQSASAAEASGGLRRCLPPGLDGSFSGSERSSPQRDGLDTSGSTGSPGAPTAARTLVSEPAAD